(19) United States
(12) Patent Application Publication
(10) Pub. No.: US 2004/0071644 A1
(43) Pub. Date: Apr. 15, 2004

(54) Nail varnish

(76) Inventors: Nathali Mougin, Paris (FR); Bertrand Lion, Luzarches (FR); Marco Vicic, Bry S/Marne (FR); Colette Cazeneuve, Paris (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/612,920

(22) Filed: Jul. 7, 2003

Related U.S. Application Data
(60) Provisional application No. 60/401,026, filed on Aug. 6, 2002.

(30) Foreign Application Priority Data
Jul. 8, 2002 (FR) 02 08554

(51) Int. Cl. A61K 7/04

(52) U.S. Cl. 424/61

(57) ABSTRACT

Disclosed herein is a nail varnish composition comprising, in a cosmetically acceptable medium, at least one film-forming polymer, wherein the varnish composition is capable of forming a film having an adherence, measured according to the ASTM D 3359-7 standard, corresponding to a percentage of detachment of less than 35%, as well as a method for making up and/or non-therapeutic caring of nails comprising applying to the nails the nail varnish composition.

This application claims benefit of U.S. Provisional Application No. 60/401,026, filed Aug. 6, 2002,

Disclosed herein is a nail varnish composition comprising at least one film-forming polymer having good adherence properties. Further disclosed herein is a method for making up and/or caring for nails.

The nail varnish composition may be used as a varnish base, as a make-up product for the nails, as a finishing composition, also called top coat, to be applied over the make-up product for the nails, and/or as a cosmetic care product for the nails. The nail varnish composition may be applied to the nails of human beings or to false nails.

The nail varnish compositions comprise, in a known manner, a film-forming polymer in an organic solvent medium or an aqueous medium. The varnish composition can form, after drying, a colored or colorless film on the nails and thus can make it possible to beautify or protect the nails against external attacks such as rubbing or scratches. However, known nail varnishes may frequently exhibit poor staying power over time: the film deteriorates, such as through flaking or detachment, after one or two days. Such a deterioration often occurs at the tip of the nail. When the varnish is damaged, the user then needs to remove the damaged varnish and then carry out another application of the varnish. The user can also touch up the damaged varnish by partially applying the varnish; but this type of touching up may not lead to a perfectly aesthetic make-up. If the user does nothing, the damaged varnish can impair the aesthetic appearance of the make-up and may not give good protection to the nail.

Other nail varnishes such as easily peelable varnishes or water-removable varnishes may not confer a good staying power over time either.

A need therefore exists to obtain novel nail varnishes, which can make it possible to obtain a film deposited on the nails having a satisfactory staying power over time, without exhibiting an unaesthetic defect.

As disclosed herein, a novel nail varnish composition is provided, which can have at least one good staying power property over time, such as good resistance to rubbing, to water and to flaking.

The inventors have discovered that a nail varnish exhibiting an adherence corresponding to a low percentage of detachment could make it possible to obtain a varnish having a good staying power over time.

Therefore, disclosed herein is a nail varnish composition comprising, in a cosmetically acceptable medium, at least one film-forming polymer, wherein the nail varnish composition is capable of forming a film having an adherence, measured according to the ASTM D 3359-7 standard, corresponding to a percentage of detachment of less than 35%.

Further disclosed herein is a cosmetic method for making up and/or non-therapeutic care of the nails comprising applying to the nails at least one layer of the nail varnish composition as defined above.

As disclosed herein, the expression “film-forming polymer” means a polymer capable of forming, on its own, or in the presence of a film-forming aid, a continuous and adherent film on the nail, at a temperature ranging from 20° C. to 30° C.

The adherence of the nail varnish film is determined with the aid of the cross-cut test defined in the ASTM D3359 standard.

A 50 μm thick layer is deposited, after drying for 24 hours at 23±2° C. and at 55±5% relative humidity, on a polyamide plate sold under the name nylon 6 by the company Goodfellow having a roughness ranging from 10 to 500 nm. The film is cut in the form of a cross-cut in accordance with the ASTM D3359 standard with the aid of a 6-blade comb each blade being 1 mm apart. The film is thus cut through its entire thickness. An ISO 2409 normalized adhesive paper is then applied to the cut film such that the adhesive paper is in contact with the entire surface of the cut film. The adhesive paper is then removed from the cross-cut film and the number of squares of film which were detached from the plate by the adhesive paper are determined.

The percentage of detachment of the film is then determined as described in the ASTM D3359 standard, for a mean of 10 samples.

The varnish composition disclosed herein can exhibit a percentage of detachment of less than 35%, such as less than 15%, and further such as less than 5%.

For example, the at least one film-forming polymer has a glass transition temperature (Tg) ranging from −100° C. to 100° C., such as from −50° C. to 80° C., further such as from 0° C. to +80° C., and even further such as from +20° C. to +80° C.

The measurement of the glass transition temperature (Tg) of the polymer is carried out by DMTA (Dynamical and Mechanical Temperature Analysis) as described below.

To measure the glass transition temperature (Tg) of the polymer, viscoelasticimetry tests are carried out using a DMTA apparatus from Polymer TA Instruments (model DMA2980), on a sample of polymer film about 150±50 μm thick, 5 mm wide and 10 mm long, after drying for 24 hours at 23±2° C. and 50-55% relative humidity. A tensile stress is applied to this sample. The sample is subjected to a static force of 0.01 N on which is superposed a sinusoidal displacement of ±8 μm at the frequency of 1 Hz. The work is thus done in the linear domain, at low deformation levels. This tensile stress is applied to the sample at temperatures varying from −150° C. to +220° C., with a temperature variation of 3° C. per minute.

The complex modulus E*=E′+iE″ of the test polymer is then measured as a function of the temperature.

The dynamic storage modulus E′ and the dynamic loss modulus E″, and the damping power: tgδ=E″/E′, are obtained from these measurements.

A curve of the tgδ values as a function of the temperature is then collated; this curve has at least one peak. The glass transition temperature Tg of the polymer corresponds to the temperature at which the summit of this peak is located.

When the curve has at least 2 peaks (in this case, the polymer has at least 2 Tg), the temperature for which the curve has the peak with the highest amplitude (i.e., corresponding to the highest tgδ value; in this case, only the “predominant” Tg is considered as Tg value for the test polymer) is taken as Tg value for the test polymer.

In one embodiment, the nail varnish composition disclosed herein is capable of forming a film having a damping power tgδ ranging from 0.5 to 1.6, and such as from 0.8 to 1.4.

For example, the nail varnish disclosed herein is capable of forming a film having a storage modulus E′ of less than 300 MPa, such as less than 100 MPa, and further such as less than 80 MPa.

The storage modulus E′ and the damping power are determined according to the viscoelastimetry test protocol described above but carried out at the constant temperature of 23° C. and the sample being subjected to a static force of 0.01 N on which is superposed a sinusoidal displacement of ±8 μm at the frequency of 20 Hz.

In one embodiment, the at least one film-forming polymer is insoluble in water at 25° C., i.e., it is soluble at less than 1% by weight in water at 25° C. (solubility of less than 1% by weight). The at least one film-forming polymer can also be, for example, soluble in at least one organic solvent, such as ethyl acetate and butyl acetate, i.e., it is soluble at more than 90% by weight in at least one organic solvent at 25° C. (solubility greater than 90% by weight at 25° C.).

The at least one film-forming polymer has, for example, a number-average molecular weight of less than or equal to 300 000, such as from 5 000 to 300 000, and further such as from 5 000 to 150 000.

The at least one film-forming polymer, which can make it possible to obtain the varnish adherence properties, may be chosen from free-radical polymers, and polycondensates. The at least one film-forming polymer can be, for example, noncrosslinked.

For example, the at least one film-forming polymer may be chosen from acrylic polymers, such as those comprising:

at least one first monomer chosen from ethylenically unsaturated monomers comprising at least one functional group chosen from carboxylic and sulphonic acid functional groups and an amide functional group, for example, chosen from (meth)acrylic acid, crotonic acid, acrylamidopropanesulphonic acid, and N-alkyl (meth)acrylamides comprising at least one alkyl group chosen from C₁-C₁₂, such as C₁-C₆, alkyl groups; ethylenically unsaturated monomers comprising at least one chlorine atom such as chlorostyrene; ethylenically unsaturated monomers comprising at least one hydroxyl group such as vinyl alcohol, and

at least one second monomer chosen from alkyl (meth)acrylates comprising at least one alkyl group chosen from C₁-C₁₈, such as C₁-C₆, alkyl groups, aryl (meth)acrylates comprising at least one aryl group chosen from C₆-C₈ aryl groups, and cycloalkyl (meth)acrylates comprising at least one group chosen from C₄-C₁₂ cycloalkyl groups.

As an example of the N-alkyl (meth)acrylamide comprising at least one alkyl group chosen from C₁-C₁₂ alkyl groups, N-tert-butyl acrylamide may be mentioned.

As examples of the alkyl (meth)acrylate comprising at least one alkyl group chosen from C₁-C₁₂ alkyl groups, methyl (meth)acrylate, ethyl (meth)acrylate, n-propyl (meth)acrylate, n-butyl (meth)acrylate, and isobutyl (meth)acrylate may be mentioned.

As an example of the aryl (meth)acrylate comprising at least one aryl group chosen from C₆-C₈ aryl groups, benzyl (meth)acrylate may be mentioned.

As examples of the cycloalkyl (meth)acrylate comprising at least one group chosen from C₄-C₁₂ cycloalkyl groups, cyclohexyl (meth)acrylate and isobornyl (meth)acrylate may be mentioned.

The acrylic polymers may further comprise at least one additional monomer chosen from vinyl esters, non-chlorinated styrene monomers, and fluorinated ethylenically unsaturated monomers.

The vinyl esters may be, for example, chosen from vinyl acetate, vinyl neodecanoate, vinyl pivalate, vinyl benzoate, and vinyl t-butylbenzoate.

As examples of the non-chlorinated styrene monomers, styrene and alphamethylstyrene may be mentioned.

As an example of the fluorinated ethylenically unsaturated monomers, trifluoroethyl methacrylate may be mentioned.

In one embodiment, the acrylic polymer is chosen from copolymers of acrylic acid and alkyl (meth)acrylate comprising at least one alkyl group chosen from C₁-C₄ alkyl groups, such as acrylic acid/methyl methacrylate/isobutyl acrylate copolymers and acrylic acid/t-butyl acrylate/isobutyl acrylate copolymers.

For example, the acrylic polymer comprises:

from 0.5% to 50% by weight of the at least one first monomer as described above, such as from 2% to 30% by weight, and further such as from 5% to 20% by weight, relative to the total weight of the monomers of the acrylic polymer,

from 40% to 99% by weight of the at least one second monomer as described above, such as from 50% to 95% by weight, and further such as from 60% to 90% by weight, relative to the total weight of the monomers of the acrylic polymer,

and optionally the balance for 100% by weight of at least one additional monomer as described above.

The at least one film-forming polymer may be moreover chosen from polycondensates, chosen, for example, from polyurethanes, polyureas, polyureaurethanes, and mixtures thereof.

The polycondensates are chosen, for example, from those formed by polycondensation:

of at least one diisocyanate chosen from linear and branched C₁-C₁₂, such as C₁-C₆, alkyl diisocyanates, C₄-C₂₀ cycloalkyl diisocyanates, and C₆-C₂₀ aryl diisocyanates;

of at least one prepolymer comprising at least two functional groups comprising at least one labile hydrogen, such as a diol and primary and secondary diamines, having a number-average molecular mass ranging from 500 to 50 000, such as from 500 to 8 000, and further such as from 1 000 to 3 000;

of at least one coupler comprising two functional groups comprising at least one labile hydrogen, such as a diol, primary and secondary diamines, and an amino alcohol, having a molecular mass of less than 500, such as greater than or equal to 50 and less than 500, and further such as greater than or equal to 75 and less than 500.

The at least one diisocyanate may be, for example, chosen from hexamethylene diisocyanate, isophorone diisocyanate, dicyclohexylmethane diisocyanate, toluene diisocyanate, diphenylmethane diisocyanate, dicyclohexylmethane diisocyanate, and tetramethylxylylene diisocyanate.

The at least one prepolymer described above may be, for example, chosen from (poly(tetramethylene oxide))diols comprising from 10 to 80 tetramethylene oxide units, polydimethylsiloxanes comprising at least one end group chosen from (C₂-C₈)alkylene-amino(C₂-C₈)alkyl groups and C₂-C₈ ω-hydroxyalkyl groups; and hydrogenated polybutadienes comprising at least one hydroxyl end group.

The at least one prepolymer is, for example, non water-soluble, i.e., the at least one prepolymer has a water-solubility of less than 1% by weight at 25° C.

The at least one coupler may be, for example, chosen from butanediol, neopentyl glycol, amino ethanol, propylene glycol, ethylene glycol, diethylene glycol, triethylene glycol and cyclohexanedimethanol.

In one embodiment, the at least one prepolymer and the at least one coupler described above are present in the at least one film-forming polymer, such as polyurethane, in an amount such that the prepolymer/coupler molar ratio ranges from 1:1 to 1:5 and the (prepolymer+coupler)/diisocyanate molar ratio ranges from 0.9:1 to 1.1:1.

For example,, the at least one film-forming polymer is such that the at least one prepolymer, the at least one diisocyanate, and the at least one coupler are present in the at least one film-forming polymer in the following molar proportion:

Prepolymer: 1;

Diisocyanate: from 2 to 6; and

Coupler: from 1 to 5.

When the (prepolymer+coupler)/diisocyanate molar ratio is less than 1, the free isocyanate groups are blocked by reaction with at least one compound comprising at least one labile hydrogen, such as ethanol.

The at least one film-forming polymer may be present in the nail varnish composition in an amount ranging from 0.1% to 60% by weight, relative to the total weight of the composition, such as from 0.1% to 40% by weight, relative to the total weight of the composition.

The nail varnish disclosed herein may further comprise at least one additional film-forming polymer, commonly called resin, such as sulphonamide resins, alkyd resins, and cellulose esters such as cellulose acetobutyrate, cellulose acetate, and cellulose acetopropionate.

The at least one additional film-forming polymer may be present in an amount ranging from 0.01% to 50% by weight, relative to the total weight of the composition, such as from 1% to 30% by weight, relative to the total weight of the composition.

The nail varnish composition disclosed herein may further comprise at least one film-forming aid for improving the film-forming properties of the varnish.

The at least one film-forming aid may be chosen from any compounds known by persons skilled in the art to be capable of fulfilling the desired function, and, for example, chosen from plasticizing agents.

The plasticizing agents may be chosen, for example, from:

citrates such as triethyl citrate, tributyl citrate, triethyl acetylcitrate, tributyl acetylcitrate, and 2-triethylhexyl acetylcitrate;

phthalates such as diethyl phthalate, dibutyl phthalate, dioctyl phthalate, dipentyl phthalate, and dimethoxyethyl phthalate; tricresyl phosphate, benzyl benzoate, tributyl phosphate, butyl acetylricinoleate, glyceryl acetylricinoleate, butyl glycolate, tributoxyethyl phosphate, triphenyl phosphate, dibutyl tartrate, camphor, glyceryl triacetate, N-ethyl-o,n-ptoluenesulphonamide, and mixtures thereof.

The at least one film-forming aid, such as the plasticizing agent, may be present in the composition in an amount ranging from 0.01% to 10% by weight, relative to the total weight of the composition, such as from 0.1% to 5% by weight, relative to the total weight of the composition. In one embodiment, the plasticizer is present in the composition in a film-forming polymer/plasticizer weight ratio ranging from 1.5:1 to 3:1.

The composition disclosed herein may comprise an aqueous medium or an organic solvent medium. In one embodiment, an organic solvent medium is used. The organic solvent medium may be, for example, anhydrous.

The aqueous medium of the composition comprises water. The water content in the composition may range from 10% to 95% by weight, relative to the total weight of the composition, such as from 40% to 90% by weight, and further such as from 60% to 85% by weight, relative to the total weight of the composition.

The varnish composition may also comprise at least one water-miscible solvent chosen, for example, from lower monoalcohols comprising from 1 to 5 carbon atoms, glycols comprising from 2 to 8 carbon atoms, C₃-C₄ ketones, C₂-C₄ aldehydes, for example, in an amount ranging from 0.1% to 15% by weight, relative to the total weight of the composition.

When the varnish composition comprises an aqueous medium, the at least one film-forming polymer is present in the form of solid particles dispersed in the aqueous medium. Such a dispersion is known by the name of latex or pseudolatex and may be prepared according to techniques well known to persons skilled in the art.

The organic solvent medium of the composition may comprise at least one organic solvent chosen, for example, from:

ketones which are liquid at room temperature, such as methyl ethyl ketone, methyl isobutyl ketone, diisobutyl ketone, isophorone, cyclohexanone, and acetone;

alcohols which are liquid at room temperature, such as ethanol, isopropanol, n-propanol, n-butanol, diacetone alcohol, 2-butoxyethanol, and cyclohexanol;

glycols which are liquid at room temperature, such as ethylene glycol, propylene glycol, pentylene glycol, and glycerol;

propylene glycol ethers which are liquid at room temperature, such as propylene glycol monomethyl ether, propylene glycol monomethyl ether acetate, and dipropylene glycol mono-n-butyl ether;

short-chain esters (having from 3 to 8 carbon atoms in total) such as ethyl acetate, methyl acetate, propyl acetate, n-butyl acetate, and isopentyl acetate;

ethers which are liquid at room temperature, such as diethyl ether, dimethyl ether, and dichlorodiethyl ether;

alkanes which are liquid at room temperature such as decane, heptane, dodecane, isododecane, and cyclohexane;

aromatic cyclic compounds which are liquid at room temperature, such as toluene and xylene; and

aldehydes which are liquid at room temperature, such as benzaldehyde and acetaldehyde.

The content of the at least one organic solvent in the composition may range, for example, from 10% to 95% by weight, relative to the total weight of the composition, such as from 40% to 90% by weight, and further such as from 60% to 85% by weight, relative to the total weight of the composition.

The varnish composition may comprise at least one thickening agent, for example, to confer on the composition a consistency allowing good application of the composition to the nails. The at least one thickening agent is, for example, chosen from organic solvent thickeners chosen, for example, from hydrophobic silicas, such as those described in the document EP-A-898960, and for example marketed under the references “AEROSIL R812®” by the company Degussa, “CAB-O-SIL TS-530®”, “CAB-O-SIL TS-610®”, “CAB-O-SIL TS-720®” by the company Cabot, “AEROSIL R972®”, “AEROSIL R974®” by the company Degussa; clays such as montmorillonite, stearalkonium hectorite, and stearalkonium bentonite; alkyl ethers of polysaccharides (such as those whose alkyl group comprises from 1 to 24 carbon atoms, such as from 1 to 10, and further such as from 1 to 6, and even further such as from 1 to 3 carbon atoms) such as those described in the document EP-A-898958, and for example marketed under the names “N-HANCE-AG 200®” and “N-HANCE AG 50®” by the company Aqualon.

The at least one thickening agent may be present in the composition disclosed herein in an amount ranging from 0.05% to 10% by weight, relative to the total weight of the composition, such as from 0.1% to 3% by weight, relative to the total weight of the composition.

The composition may also comprise at least one coloring matter chosen, for example, from fat-soluble colorants, pigments, pearlescent agents and glitter.

The fat-soluble colorants may be, for example, chosen from Sudan red, DC Red 17, DC Green 6, β-carotene, soybean oil, Sudan brown, DC Yellow 11, DC Violet 2, DC Orange 5, and quinoline yellow. The fat-soluble colorant may be present in the composition in an amount ranging, for example, from 0.01% to 6% by weight, relative to the total weight of the composition, such as from 0.01% to 3% by weight, relative to the total weight of the composition.

The at least one coloring matter chosen from pigments, pearlescent agents and glitter may be present in the composition, such as the base and/or top composition, in an amount ranging, for example, from 0.01% to 25% by weight, relative to the total weight of the composition, such as from 0.01% to 15% by weight, relative to the total weight of the composition. The pigments can be chosen from inorganic pigments and organic pigments. The inorganic pigmentscan be chosen, for example, from titanium, zirconium and cerium oxides, zinc, iron and chromium oxides and ferric blue. The organic pigments may be chosen, for example, from carbon black, barium, strontium, calcium and aluminium lacquers. Among the pearlescent agents, mica coated with titanium oxide, with iron oxide, with a natural pigment or with bismuth oxychloride, such as colored mica-titanium, may be, for example, used.

The varnish composition disclosed herein, such as the base and/or top composition, may further comprise at least one cosmetic additive chosen from those known to persons skilled in the art as being capable of being incorporated into such a composition, such as fillers, spreading agents, wetting agents, dispersing agents, antifoams, preservatives, UV-screening agents, active agents, surfactants, moisturizing agents, perfumes, neutralizers, stabilizers, and antioxidants. Of course, persons skilled in the art will be careful to choose this or these optional additional compound(s), and/or their quantity, such that the advantageous properties of the corresponding composition disclosed herein are not, or not substantially, impaired by the envisaged addition.

The invention is illustrated in greater detail in the following examples, which are non-limiting in nature.

### EXAMPLE 1:

a) The following film-forming polymer was prepared:

Methyl methacrylate/isobutyl acrylate/acrylic acid copolymer (55/25/20 by weight).

55 g of methyl methacrylate, 25 g of isobutyl acrylate, 20 g of acrylic acid, 0.6 g of initiator bis(2-ethylhexanoylperoxy)-2,5-dimethylhexane sold under the name Trigonox 141 by the company AKZO CHEMIE, 70 g of butyl acetate and 30 g of ethyl acetate were introduced under a nitrogen atmosphere into a 500 ml reactor equipped with central stirring and a condenser.

The medium was stirred at room temperature and then heated at 90° C. for 1 hour. The reaction was allowed to progress at 90° C. for 18 hours.

The polymer was finally obtained in the butyl acetate/ethyl acetate mixture at a concentration of 50% by weight.

The polymer has the following characteristics: Number-average molecular weight: 6 500 g/mol Glass transition temperature: +65° C.

b) A nail varnish having the following composition was prepared:

The polymer solution obtained in a butyl acetate/ethyl acetate 70/30 by weight mixture was diluted to a concentration by weight of polymer equal to 25%.

The solution obtained was then applied to the nails. A varnish was obtained, after drying, whose film has the following characteristics, measured according to the protocols described above:

Percentage of detachment: 30%

Damping power tgδ: 0.8

Storage modulus E′: 100 MPa

The film exhibits a good staying power over time, such as a good resistance to flaking.

### Example 2:

a) The following film-forming polymer was prepared:

Isobutyl acrylate/tert-butyl acrylate/acrylic acid copolymer (50/40/10 by weight).

50 g of isobutyl acrylate, 40 g of tert-butyl acrylate, 10 g of acrylic acid, 0.2 g of initiator bis(2-ethylhexanoylperoxy)-2,5-dimethylhexane and 100 g of ethanol were introduced, under a nitrogen atmosphere, into a 500 ml reactor equipped with central stirring and a condenser.

The reaction medium was stirred at room temperature and then heated at 77° C. while allowing the reaction to proceed for 10 hours.

The polymer in solution obtained was precipitated from petroleum ether and then dried in an oven at 40° C. under vacuum in the presence of phosphoric anhydride.

The polymer has the following characteristics:

Number-average molecular weight: 51 500 g/mol

Glass transition temperature: +40° C.

b) A nail varnish having the following composition was prepared:

25 g of polymer obtained were dissolved in 70 g of a butyl acetate/ethyl acetate 70/30 by weight mixture.

The solution obtained was then applied to the nails. A varnish was obtained, after drying, whose film has the following characteristics, measured according to the protocols described above:

Percentage of detachment: ranging from 5% to 15%

Damping power tgδ: 1.3

Storage modulus E′: 26 MPa

The film exhibits a good staying power over time, such as a good resistance to flaking.

What is claimed is:

1. A nail varnish composition comprising, in a cosmetically acceptable medium, at least one film-forming polymer, wherein the nail varnish composition is capable of forming a film having an adherence, measured according to the ASTM D 3359-7 standard, corresponding to a percentage of detachment of less than 35%.

2. The nail varnish composition according to claim 1, wherein the percentage of detachment is less than 15%.

3. The nail varnish composition according to claim 2, wherein the percentage of detachment is less than 5%.

4. The nail varnish composition according to claim 1, wherein the at least one film-forming polymer has a glass transition temperature ranging from −100° C. to +100° C.

5. The nail varnish composition according to claim 4, wherein the at least one film-forming polymer has a glass transition temperature ranging from −50° C. to +80° C.

6. The nail varnish composition according to claim 5, wherein the at least one film-forming polymer has a glass transition temperature ranging from 0° C. to +80° C.

7. The nail varnish composition according to claim 6, wherein the at least one film-forming polymer has a glass transition temperature ranging from +20° C. to +80° C.

8. The nail varnish composition according to claim 1, wherein the nail varnish composition is capable of forming a film having a damping power tgδ ranging from 0.5 to 1.6.

9. The nail varnish composition according to claim 8, wherein the nail varnish composition is capable of forming a film having a damping power ranging from 0.8 to 1.4.

10. The nail varnish composition according to claim 1, wherein the nail varnish composition is capable of forming a film having a storage modulus E′ of less than 300 MPa.

11. The nail varnish composition according to claim 10, wherein the nail varnish composition is capable of forming a film having a storage modulus E′ of less than 150 MPa.

12. The nail varnish composition according to claim 11, wherein the nail varnish composition is capable of forming a film having a storage modulus E′ of less than 80 MPa.

13. The nail varnish composition according to claim 1, wherein the at least one film-forming polymer is chosen from acrylic polymers, polyurethanes, polyureas, and polyurea-urethanes.

14. The nail varnish composition according to claim 13, wherein the at least one film-forming polymer is chosen from acrylic polymers comprising:
at least one first monomer chosen from ethylenically unsaturated monomers comprising at least one functional group chosen from carboxylic and sulphonic acid functional groups and an amide functional group; ethylenically unsaturated monomers comprising at least one chlorine atom; ethylenically unsaturated monomers comprising at least one hydroxyl group,
at least one second monomer chosen from alkyl (meth)acrylates comprising at least one alkyl group chosen from C₁-C₁₈ alkyl groups, aryl (meth)acrylates comprising at least one aryl group chosen from C₆-C₈ aryl groups, and cycloalkyl (meth)acrylates comprising at least one group chosen from C₄-C₈ cycloalkyl groups.

15. The nail varnish composition according to claim 14, wherein the at least one first monomer is chosen from (meth)acrylic acid, crotonic acid, acrylamidopropanesulphonic acid, N-alkyl (meth)acrylamides comprising at least one alkyl group chosen from C₁-C₁₂ alkyl groups, chlorostyrene, and vinyl alcohol.

16. The nail varnish composition according to claim 14, wherein the at least one first monomer is chosen from (meth)acrylic acid, crotonic acid, acrylamidopropanesulphonic acid, N-tert-butyl acrylamide, chlorostyrene, and vinyl alcohol.

17. The nail varnish composition according to claim 14, wherein the at least one second monomer is chosen from methyl (meth)acrylate, ethyl (meth)acrylate, n-propyl (meth)acrylate, n-butyl (meth)acrylate, isobutyl (meth)acrylate, cyclohexyl (meth)acrylate, and isobornyl (meth)acrylate.

18. The nail varnish composition according to claim 14, wherein the acrylic polymers comprise at least one additional monomer chosen from vinyl esters, non-chlorinated styrene monomers, and fluorinated ethylenically unsaturated monomers.

19. The nail varnish composition according to claim 18, wherein the at least one additional monomer is chosen from vinyl acetate, vinyl neodecanoate, vinyl pivalate, vinyl benzoate, vinyl t-butylbenzoate, styrene, alpha-methylstyrene, and trifluoroethyl methacrylate.

20. The nail varnish composition according to claim 18, wherein the acrylic polymers comprise:
from 0.5% to 50% by weight of the at least one first monomer, relative to the total weight of the monomers of the acrylic polymer,
from 40% to 99% by weight of the at least one second monomer, relative to the total weight of the monomers of the acrylic polymer,
and optionally the balance for 100% of the at least one additional monomer.

21. The nail varnish composition according to claim 18, wherein the acrylic polymers comprise:
from 2% to 30% by weight of the at least one first monomer, relative to the total weight of the monomers of the acrylic polymer,
from 50% to 95% by weight of the at least one second monomer, relative to the total weight of the monomers of the acrylic polymer,
and optionally the balance for 100% of the at least one additional monomer.

22. The nail varnish composition according to claim 18, wherein the acrylic polymers comprise:
from 5% to 20% by weight of the at least one first monomer, relative to the total weight of the monomers of the acrylic polymer,
from 60% to 90% by weight of the at least one second monomer, relative to the total weight of the monomers of the acrylic polymer, and optionally the balance for 100% of the at least one additional monomer.

23. The nail varnish composition according to claim 14, wherein the acrylic polymers are chosen from copolymers of acrylic acid and/or tert-butyl acrylamide and alkyl (meth)acrylate comprising at least one alkyl group chosen from C₁-C₄ alkyl groups.

24. The nail varnish composition according to claim 1, wherein the at least one film-forming polymer is chosen from copolymers of acrylic acid and alkyl (meth)acrylate comprising at least one alkyl group chosen from C₁-C₄ alkyl groups.

25. The nail varnish composition according to claim 13, wherein the acrylic polymers are chosen from acrylic acid/methyl methacrylate/isobutyl acrylate copolymers and acrylic acid/t-butyl acrylate/isobutyl acrylate copolymers.

26. The nail varnish composition according to claim 1, wherein the at least one film-forming polymer is chosen from polycondensates formed by polycondensation:
of at least one diisocyanate chosen from linear and branched C₁-C₁₂ alkyl diisocyanates, C₄-C₂₀ cycloalkyl diisocyanates, and C₆-C₂₀ aryl diisocyanates;
of at least one prepolymer comprising at least two functional groups comprising at least one labile hydrogen, having a number-average molecular mass ranging from 500 to 50 000; and
of at least one coupler comprising two functional groups comprising at least one labile hydrogen, having a molecular mass of less than 500.

27. The nail varnish composition according to claim 26, wherein in the at least one prepolymer, the at least two functional groups comprising at least one labile hydrogen are chosen from diols and primary and secondary diamines.

28. The nail varnish composition according to claim 26, wherein the at least one prepolymer has a number-average molecular mass ranging from 500 to 8,000.

29. The nail varnish composition according to claim 28, wherein the at least one prepolymer has a number-average molecular mass ranging from 1,000 to 3,000.

30. The nail varnish composition according to claim 26, wherein in the at least one coupler, the two functional groups comprising at least one labile hydrogen are chosen from diols, primary and secondary diamines, and amino alcohols.

31. The nail varnish composition according to claim 26, wherein the at least one coupler has a molecular mass of greater than or equal to 50 and less than 500.

32. The nail varnish composition according to claim 31, wherein the at least one coupler has a molecular mass of greater than or equal to 75 and less than 500.

33. The nail varnish composition according to claim 26, wherein the at least one diisocyanate is chosen from hexamethylene diisocyanate, isophorone diisocyanate, dicyclohexylmethane diisocyanate, toluene diisocyanate, diphenylmethane diisocyanate, dicyclohexylmethane diisocyanate, and tetramethylxylylene diisocyanate.

34. The nail varnish composition according to claim 26, wherein the at least one prepolymer is chosen from (poly(tetramethylene oxide))diols comprising from 10 to 80 tetramethylene oxide units; polydimethylsiloxanes comprising at least one end group chosen from (C₂-C₈)alkyleneamino(C₂-C₈)alkyl groups and C₂-C₈ (ω-hydroxyalkyl groups; and hydrogenated polybutadienes comprising at least one hydroxyl end group.

35. The nail varnish composition according to claim 26, wherein the at least one prepolymer is non water-soluble.

36. The nail varnish composition according to claim 26, wherein the at least one coupler is chosen from butanediol, neopentyl glycol, amino ethanol, propylene glycol, ethylene glycol, diethylene glycol, triethylene glycol, and cyclohexanedimethanol.

37. The nail varnish composition according to claim 26, wherein the at least one prepolymer and the at least one coupler are present in the at least one film-forming polymer in an amount such that the prepolymer/coupler molar ratio ranges from 1:1 to 1:5 and the (prepolymer+coupler)/diisocyanate molar ratio ranges from 0.9:1 to 1.1:1.

38. The nail varnish composition according to claim 37, wherein the at least one film-forming polymer is chosen from polyurethanes.

39. The nail varnish composition according to claim 26, wherein the at least one film-forming polymer is such that the at least one prepolymer, the at least one diisocyanate, and the at least one coupler are present in the at least one film-forming polymer in the following molar proportion:
the at least one prepolymer: 1;
the at least one diisocyanate: from 2 to 6; and
the at least one coupler: from 1 to 5.

40. The nail varnish composition according to claim 26, wherein when the (prepolymer +coupler)/diisocyanate molar ratio is less than 1, the free isocyanate groups are blocked by reaction with at least one compound comprising at least one labile hydrogen.

41. The nail varnish composition according to claim 40, wherein the at least one compound comprising at least one labile hydrogen is ethanol.

42. The nail varnish composition according to claim 1, wherein the at least one film-forming polymer has a number-average molecular weight of less than or equal to 300 000.

43. The nail varnish composition according to claim 42, wherein the at least one film-forming polymer has a number-average molecular weight ranging from 10 000 to 150 000.

44. The nail varnish composition according to claim 1, wherein the at least one film-forming polymer is present in an amount ranging from 0.1% to 60% by weight, relative to the total weight of the composition.

45. The nail varnish composition according to claim 44, wherein the at least one film-forming polymer is present in an amount ranging from 0.1% to 40% by weight, relative to the total weight of the composition.

46. The nail varnish composition according to claim 1, further comprising at least one additional film-forming polymer chosen from sulphonamide resins, alkyd resins, and cellulose esters.

47. The nail varnish composition according to claim 1, further comprising at least one plasticizing agent.

48. The nail varnish composition according to claim 1, comprising at least one organic solvent medium.

49. The nail varnish composition according to claim 48, wherein the at least one organic solvent medium is anhydrous.

50. The nail varnish composition according to claim 48, wherein the at least one organic solvent medium comprises at least one organic solvent chosen from ketones; alcohols, glycols, propylene glycol ethers, short-chain esters, ethers, alkanes, aromatic cyclic compounds, and aldehydes.

51. The nail varnish composition according to claim 1, comprising at least one aqueous medium.

52. The nail varnish composition according to claim 1, further comprising at least one cosmetic additive chosen from thickening agents, coloring matters, fillers, spreading agents, wetting agents, dispersing agents, antifoams, preservatives, UV-screening agents, active agents, surfactants, moisturizing agents, perfumes, neutralizers, stabilizers, and antioxidants.

53. A cosmetic method for making up and/or non-therapeutic care of nails, comprising applying to the nails at least one layer of a nail varnish comprising, in a cosmetically acceptable medium, at least one film-forming polymer, wherein the nail varnish composition is capable of forming a film having an adherence, measured according to the ASTM D 3359-7 standard, corresponding to a percentage of detachment of less than 35%.
